(19) United States
(12) Patent Application Publication
(10) Pub. No.: US 2023/0159202 A1
(43) Pub. Date: May 25, 2023

(54) METHOD FOR MAKING A PRESERVATIVE-FREE PACKAGED OPHTHALMIC DEVICE PRODUCT

(71) Applicant: Bausch + Lomb Ireland Limited, Dublin (IE)

(72) Inventor: David J. Heiler, Avon, NY (US)

(21) Appl. No.: 17/533,978

(22) Filed: Nov. 23, 2021

(51) Int. Cl. B65B 25/00; B65B 55/14; B65B 55/22

(52) U.S. Cl. CPC B65B 25/008; B65B 55/14; B65B 55/22

(57) ABSTRACT

A preservative-free, sterile packaged ophthalmic device product is disclosed. The preservative-free, sterile packaged ophthalmic device product is obtained by (a) adding at least one polyquaternium polymer to an aqueous packaging solution for preventing antimicrobial contamination during storage; (b) packaging the aqueous packaging solution and an ophthalmic device in a manner preventing contamination of the ophthalmic device by one or more microorganisms; and (c) heat sterilizing the aqueous packaging solution and the ophthalmic device, whereby the at least one polyquaternium polymer decomposes during the heat sterilizing to obtain the preservative-free packaged ophthalmic device product.

### BACKGROUND

Blister-packs and glass vials are typically used to individually package each soft contact lens for sale to a customer. The blister-packs and glass vials contain a packaging solution for storing the lens. Borate buffers have typically been used in the packaging solutions due to their sufficient buffering capability and ability to resist microbial growth. Presently, the limit of the amount of a borate buffer in aqueous packaging solutions is below 0.3 wt. %. It is believed however that at these levels a borated buffer-containing packaging solution could not inhibit microbial growth.

Accordingly, it would be desirable to provide an improved packaging system for ophthalmic devices such as a contact lens which contains a borated buffer-free packaging solution that can inhibit microbial growth during the manufacturing process.

### SUMMARY

In accordance with one exemplary embodiment, a method is provided comprising (a) adding at least one polyquaternium polymer to an aqueous packaging solution for preventing antimicrobial contamination during storage; (b) packaging the aqueous packaging solution and an ophthalmic device in a manner preventing contamination of the ophthalmic device by microorganisms; and (c) heat sterilizing the aqueous packaging solution and the ophthalmic device, whereby the at least one polyquaternium polymer decomposes during the heat sterilizing to obtain a preservative-free packaged ophthalmic device product.

In accordance with another exemplary embodiment, a method of making a preservative-free, sterile packaged ophthalmic device product is provided, the method comprising: (a) immersing an ophthalmic device in an aqueous packaging solution comprising at least one polyquaternium polymer antimicrobial agent, wherein the aqueous packaging solution has an osmolality of at least about 150 mOsm/kg and a pH in the range of about 6 to about 9; (b) packaging the aqueous packaging solution and the ophthalmic device in a manner preventing contamination of the ophthalmic device by microorganisms; and (c) heat sterilizing the aqueous packaging solution and the ophthalmic device to obtain a preservative-free, sterile package ophthalmic device product.

### DETAILED DESCRIPTION

Illustrative embodiments provided herein are directed to at least preventing antimicrobial contamination of an aqueous packaging solution containing at least one polyquaternium polymer during manufacturing of the ophthalmic device and thereafter obtaining a preservative-free, sterile package ophthalmic device product by heat sterilizing, e.g., autoclaving, the aqueous packaging solution and ophthalmic device whereby the at least one polyquaternium polymer breaks down during autoclaving. For example, by adding at least one polyquaternium polymer to an aqueous packaging solution as described herein, the polyquaternium polymer is believed to inhibit microbial growth during the ophthalmic device (e.g., contact lens) manufacturing process. In addition, during the sterilization process (such as autoclaving) of packaging an ophthalmic device in the aqueous packaging solution, the polyquaternium polymer decomposes into its monomeric and oligomeric species such as initial monomeric and oligomeric species with tertiary amine and hydroxyl end groups, thereby providing a preservative-free, sterile packaged ophthalmic device product.

By “ophthalmic device manufacturing” or “lens manufacturing” is meant storing the aqueous packaging solution under conditions such that, for example, the solutions come into contact with non-sterile items such as carboys, vats, hoses, blister packages, etc. for a time period up to about 30 days.

In one illustrative embodiment, the aqueous packaging solution will contain at least one polyquaternium polymer. The at least one polyquaternium polymer can have a quaternary-amine-functional repeat unit ranging from about 30 units to about 50,000 units. In one exemplary embodiment, the at least one polyquaternium polymer can have a quaternary-amine-functional repeat unit ranging from about 50 units to about 2,000 units. A “quaternary-amine-functional repeat unit” is herein understood to mean that the repeat unit comprises a quaternary-amine group in which a positively charged nitrogen atom is covalently bonded to four radicals (no hydrogen atoms) and ionically bonded to a negatively charged counterion such as a chloride.

The at least one polyquaternium polymer can have a weight average molecular weight Mw of about 3,000 to about 5,000,000. In one exemplary embodiment, the at least one polyquaternium polymer can have a weight average molecular weight Mw of about 5,000 to about 500,000. In one exemplary embodiment, the at least one polyquaternium polymer can have a weight average molecular weight Mw of about 5,000 to about 200,000. In one exemplary embodiment, the at least one polyquaternium polymer can have a weight average molecular weight Mw of about 5,000 to about 50,000. In one exemplary embodiment, the at least one polyquaternium polymer can have a weight average molecular weight Mw of about 5,000 to about 30,000.

The polyquaternium polymers useful herein may include, but are not limited to, copolymers in which the quaternary-amine-functional repeat units are derived from one or more of the following kinds of monomers: N,N-dimethyl-N-ethyl-aminoethyl acrylate and methacrylate, 2-methacryloxyethyltrimethylammonium, N-(3-methacrylamidopropyl)-N,N,N-trimethylammonium, 1-vinyl and 3-methyl-1-vinylimidazole, N-(3-acrylamido-3-methylbutyl)-N,N,N-trimethyl ammonium, N-(3-methacryloyloxy-2-hydroxypropyl)-N,N,N-trimethylammonium, their halides or other salt forms, and derivatives thereof, for example, involving the substitution, addition, or removal of alkyl groups such as alkyl groups having 1 to 6 carbon atoms. Quaternary-amine-functional repeat units can also be obtained as a reaction product or two or more compounds, for example, by the use of a strong alkylating agent such as 1,4-dichloro-2-butene which, for example, can be reacted with 1,4-bis[dimethylaminol]-2-butene and triethanolamine to produce a polymeric polyquartenary ammonium compound. Quaternary-amine-functional repeat units can also be made from other polymers, such as by the reaction of a trimethyl amonium substituted epoxide with the hydroxy group of a hydroxyethylcellulose. Suitable quaternary-amine-functional repeat units also include those found in polymeric ionenes and the like formed by a polycondensation reaction; in such repeat units, the nitrogens of the quaternary-amines are integral to the polymeric backbone and are situated between alkylene, oxyalkylene, or other segments.

In an exemplary embodiment, the nitrogens in the quaternary-amine-functional repeat units are part of a saturated or unsaturated heterocyclic ring, such as a five- or six-membered ring. In one embodiment, the polyquaternium polymer is a copolymer of a vinylimidazolium salt or a dimethyldiallyl ammonium salt. In one embodiment, up to about 90%, e.g., about 40% to about 90% by mole, of copolymerization-compatible comonomers not having a quaternary-amine-functionality may be copolymerized with the quaternary-amine-functional comonomers. Suitable comonomers include, for example, vinylpyrrolidone, acrylic acid, alkyl methacryate, amides and amines such as acrylamide and N,N-dialkylaminoalkyl acrylate and methacrylate, hydroxyethylcellulose and copolymerization-compatible mixtures thereof. In one embodiment, an alkyl group has 1 to 6 carbon atoms.

Polyquaternium polymers as thus defined are a well-known class of polymers, many variations of which are commercially available. For example, a current CTFA International Cosmetic Ingredient Dictionary includes polyquaterniums designated as Polyquaternium-1 through Polyquaternium-68, a number of which, based on the present teachings, are useful in the illustrative embodiments described herein. The polymerization techniques for the preparation of such materials are similarly well known to those skilled in the art and many variations of such techniques are similarly in practice in commerce. New variations of such polyquaternium polymers are in continuous commercial development, for example, various polymers having different combinations of the same or similar repeat units, different relative proportions of comonomers, and/or different molecular weights are in continuous commercial development.

In one embodiment, the least one polyquaternium polymer is Polyquaternium-1. Polyquaternium-1 is either commercially available from such sources as Stepan Inc. under the tradename Onamer M® or can be synthesized by well-known methods, see, for example, U.S. Pat. No. 4,027,020, the contents of which are incorporated by reference herein. If desired, the polymer can have alternative end groups such as hydroxyallylic end groups, aminoallylic end groups and diene end groups, see, for example, U.S. Pat. No. 7,705,112, the contents of which are incorporated by reference herein.

As discussed above, the polyquaternium polymers such as Polyquaternium-1 decomposes into monomeric and oligomeric species during the sterilization process, e.g., autoclaving or heating at or above 120° C. in aqueous solutions, mainly by hydrolysis of the quaternary ammonium group. The monomeric species formed for Polyquaternium-1 include 1,4-dihydroxy-2-butene, bis-(N,N-dimethyl)-1,4-butenediame and triethanolamine as well as low molecular weight oligomers with endgroups described in U.S. Pat. No. 7,705,112. One of the monomers used to synthesize Polyquaternium-1, 1,4-dichloro-2-butene, cannot reform from PQ-1 hydrolysis since the chloroalkyl groups are replaced by hydroxyalkyl groups. This is advantageous herein as the thermally degraded Polyquaternium-1 products do not have antimicrobial preservative efficacy and thus not expected to have any adverse lens interactions or other effects at concentrations less than, for example, about 10 ppm.

The polyquaternium polymer suitably includes an ophthalmologically suitable anionic organic or inorganic counterion. In an illustrative embodiment, a preferred counterion is chloride.

In certain embodiments, the cationic oligomer or polymer is characterized by a charge density that may be determined by methods known in the art, such as colloidal titration. In one embodiment, the charge density of the cationic oligomer or polymer is at least about 0.1 meq/g, in another embodiment at least about 2.5 meq/g, and in yet another embodiment, at least about 5 meq/g.

The polyquaternium polymer can be present in the aqueous packaging solution in an amount of from about 0.1 ppm to about 100 ppm. In one embodiment, the polyquaternium polymer can be present in the aqueous packaging solution in an amount of from about 0.5 ppm to about 20 ppm.

The aqueous packaging solution can further contain one or more additional additives. For example, in one illustrative embodiment, the aqueous packaging solution can further contain one or more of (a) one or more osmoprotectants, (b) one or more poloxamer comfort agents and (c) one or more demulcent polyols. Suitable osmoprotectants include, for example, polyols, amino acids and methylamine-containing compounds. Suitable polyols for use herein have the formula R″(OH)_{y} where R″ is a hydrocarbon radical and y is an integer representing the number of hydroxy radicals and has a value of, for example, from 3 to about 12. The polyols may contain less than about 12 carbon atoms. Examples of suitable polyols include, but are not limited to, alkylene glycols and poly(oxyalkylene) glycols, e.g., ethylene glycol, di(ethylene glycol), tri(ethylene glycol), di(propylene glycol), tri(butylene glycol), penta(ethylene glycol), and other poly(oxyalkylene) glycols formed by the condensation of two or more moles of ethylene glycol, propylene glycol, octylene glycol, or a like glycol having up to 12 carbon atoms in the alkylene radical. Other examples of suitable polyols include, but are not limited to, pentaerythritol, erythritol, sucrose, trehalose, xylitol, raffinose, raffinose/galactinol and the like. In one embodiment, the polyol is erythritol.

Suitable amino acids include, for example, amino acids occurring in the natural collagen of the cornea, such as betaine, glycine betaine, glycine, diglycine, proline, glutamine, alanine, arganine, asparagine, lysine, leucine, serine and isoleucine.

Suitable methylamine-containing compounds include, for example, sarcosine, trimethylamine N-oxide, betaine, glycine betaine and L-carnitine.

The amount of the one or more osmoprotectants employed in an aqueous packaging solution for storing an ophthalmic device in a packaging system described herein is an amount effective to improve the properties of the ophthalmic device. It is believed these osmoprotectants enhance initial and extended comfort when a contact lens, packaged in the solution and then removed from the packaging system, is placed on the eye for wearing. In one embodiment, the concentration of the one or more osmoprotectants present in the aqueous packaging solution will range from about 0.01% to about 10% w/w. In one embodiment, the concentration of the one or more osmoprotectants present in the aqueous packaging solution will range from about 0.1% to about 10% w/w.

The aqueous packaging solution can further contain one or more poloxamer comfort agents. A representative example of a suitable poloxamer comfort agent is a poloxamer block copolymer. One specific class of poloxamer block copolymers are those available under the trademark Pluronic (BASF Wyandotte Corp., Wyandotte, Mich.). Poloxamers include Pluronics and reverse Pluronics. Pluronics are a series of ABA block copolymers composed of poly(ethylene oxide)-poly(propylene oxide)-poly(ethylene oxide) blocks as generally represented in Formula I:

HO(C₂H₄O)ₐ(C₃H₆O)b(C₂H₄O)ₐH (I)

wherein a is independently at least 1 and b is at least 1.

Reverse Pluronics are a series of BAB block copolymers, respectively composed of poly(propylene oxide)-poly(ethylene oxide)-poly(propylene oxide) blocks as generally represented in Formula II:

HO(C₃H₆O)_{b}(C₂H₄O)ₐ(C₃H₆O)_{b}H (II)

wherein a is at least 1 and b is independently at least 1. The poly(ethylene oxide), PEO, blocks are hydrophilic, whereas the poly(propylene oxide), PPO, blocks are hydrophobic in nature. The poloxamers in each series have varying ratios of PEO and PPO which ultimately determines the hydrophilic-lipophilic balance (HLB) of the material, i.e., the varying HLB values are based upon the varying values of a and b, a representing the number of hydrophilic poly(ethylene oxide) units (PEO) being present in the molecule and b representing the number of hydrophobic poly(propylene oxide) units (PPO) being present in the molecule. In one embodiment, the poloxamer will have an HLB ranging from about 5 to about 24. In one embodiment, the poloxamer will have an HLB ranging from about 1 to about 5.

Poloxamers and reverse poloxamers have terminal hydroxyl groups that can be terminal functionalized. An example of a terminal functionalized poloxamer and as discussed herein is poloxamer dimethacrylate (e.g., Pluronic® F127 dimethacrylate) as disclosed in U.S. Patent Application Publication No. 2003/0044468 and U.S. Pat. No. 9,309,357, the contents of which are incorporated by reference herein. Other examples include glycidyl-terminated copolymers of polyethylene glycol and polypropylene glycol as disclosed in U.S. Pat. No. 6,517,933, the contents of which are incorporated by reference herein.

The poloxamer is functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomeric mixture. The term block copolymer as used herein shall be understood to mean a poloxamer as having two or more blocks in their polymeric backbone(s).

In one embodiment, the one or more poloxamer comfort agents are present in the aqueous packaging solution in an amount ranging from about 0.001 to about 5.0 wt. %, based on the total weight of the aqueous packaging solution. In another illustrative embodiment, the one or more poloxamer comfort agents are present in the aqueous packaging solution in an amount ranging from about 0.01 to about 1.0 wt. %, based on the total weight of the aqueous packaging solution.

The aqueous packaging solution can further contain one or more polyol demulcents. Suitable polyols for use herein have the formula R″(OH)_{y} where R″ is a hydrocarbon radical and y is an integer representing the number of hydroxy radicals and has a value of from 2 to 3. The polyols may contain less than about 12 carbon atoms. Representative examples of polyol demulcents include glycerol, propylene glycol, polyethylene glycol 300, polyethylene glycol 400, and polysorbate 80.

In one embodiment, the one or more polyol demulcents are present in the aqueous packaging solution in an amount ranging from about 0.01 to about 10.0 wt. %, based on the total weight of the aqueous packaging solution. In another illustrative embodiment, the one or more polyol demulcents are present in the aqueous packaging solution in an amount ranging from about 0.1 to about 2.0 wt. %, based on the total weight of the aqueous packaging solution.

The aqueous packaging solution may further contain one or more poloxamines. While the poloxamers and reverse poloxamers are considered to be difunctional molecules (based on the terminal hydroxyl groups), the poloxamines are in a tetrafunctional form, i.e., the molecules are tetrafunctional block copolymers terminating in primary hydroxyl groups and linked by a central diamine. One specific class of poloxamine block copolymers are those available under the trademark Tetronic (BASF). Poloxamines include Tetronic and reverse Tetronics. Poloxamines have the following general structure of Formula III:

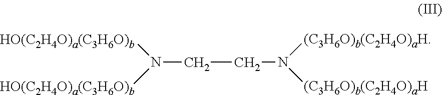

wherein a is independently at least 1 and b is independently at least 1.

The poloxamine is functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomeric mixture. The term block copolymer as used herein shall be understood to mean a poloxamine as having two or more blocks in their polymeric backbone(s).

In one embodiment, the one or more poloxamines are present in the aqueous packaging solution in an amount ranging from about 0.001 to about 5.0 wt. %, based on the total weight of the aqueous packaging solution. In another illustrative embodiment, the one or more poloxamines are present in the aqueous packaging solution in an amount ranging from about 0.1 to about 1.2 wt. %, based on the total weight of the aqueous packaging solution.

The aqueous packaging solutions of the illustrative embodiments described herein are physiologically compatible. Specifically, the solution must be “ophthalmically safe” for use with an ophthalmic device such as a contact lens, meaning that a contact lens treated with the solution is generally suitable and safe for direct placement on the eye without rinsing, that is, the solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations.

The aqueous packaging solution should also be sterile in that the absence of microbial contaminants in the product prior to release must be statistically demonstrated to the degree necessary for such products. The liquid media useful herein are selected to have no substantial detrimental effect on the lens being treated or cared for and to allow or even facilitate the present lens treatment or treatments. In one embodiment, the liquid media is aqueous-based. A particularly useful aqueous liquid medium is that derived from saline, for example, a conventional saline solution or a conventional buffered saline solution.

The pH of the packaging solutions should be maintained within the range of about 6 to about 9, or from about 6.5 to about 7.8. Suitable buffers may be added, such as boric acid, sodium borate, potassium citrate, citric acid, sodium bicarbonate, TRIS and various mixed phosphate buffers (including combinations of Na₂HPO₄, NaH₂PO₄ and KH₂PO₄) and mixtures thereof. Generally, buffers will be used in amounts ranging from about 0.05 to about 2.5 percent by weight of the solution. In one embodiment, buffers will be used in amounts ranging from about 0.1 to about 1.5 percent by weight of the solution. In one illustrative embodiment, an aqueous packaging solutions described herein contain a borate buffer, containing one or more of boric acid, sodium borate, potassium tetraborate, potassium metaborate or mixtures of the same.

Typically, the aqueous packaging solutions are also adjusted with tonicity agents, to approximate the osmotic pressure of normal lacrimal fluids which is equivalent to a 0.9 percent solution of sodium chloride or 2.5 percent of glycerol solution. The packaging solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution which will cause stinging and eye irritation.

Suitable tonicity adjusting agents include, for example, sodium and potassium chloride, dextrose, calcium and magnesium chloride and the like and mixtures thereof. These tonicity adjusting agents are typically used individually in amounts ranging from about 0.01 to about 2.5% w/v. In one embodiment, the tonicity adjusting agents are used in amounts ranging from about 0.2 to about 1.5% w/v. The tonicity agent will be employed in an amount to provide a final effective osmotic value of at least about 150 mOsm/kg. In one embodiment, the tonicity adjusting agents are used in an amount to provide a final effective osmotic value of from about 150 to about 420 mOsm/kg. In one embodiment, the tonicity adjusting agents are used in an amount to provide a final effective osmotic value of from about 150 to about 350 mOsm/kg. In one embodiment, the tonicity adjusting agents are used in an amount to provide a final effective osmotic value of from about 160 to about 320 mOsm/kg after packaging.

If desired, one or more further components can be included in the aqueous packaging solution. Such additional component or components are chosen to impart or provide at least one beneficial or desired property to the aqueous packaging solution. In general, the additional components may be selected from components which are conventionally used in one or more ophthalmic device care compositions. Suitable additional components include, for example, cleaning agents, wetting agents, nutrient agents, sequestering agents, viscosity builders, contact lens conditioning agents, antioxidants, and the like and mixtures thereof. These additional components may each be included in the aqueous packaging solutions in an amount effective to impart or provide the beneficial or desired property to the aqueous packaging solutions. For example, such additional components may be included in the aqueous packaging solutions in amounts similar to the amounts of such components used in other, e.g., conventional, contact lens care products.

Suitable sequestering agents include, for example, disodiumethylene diamine tetraacetate, alkali metal hexametaphosphate, citric acid, sodium citrate and the like and mixtures thereof.

Suitable viscosity builders include, for example, hydroxyethyl cellulose, hydroxymethyl cellulose, polyvinyl pyrrolidone, polyvinyl alcohol and the like and mixtures thereof.

Suitable antioxidants include, for example, sodium metabisulfite, sodium thiosulfate, N-acetylcysteine, butylated hydroxyanisole, butylated hydroxytoluene and the like and mixtures thereof.

The aqueous packaging solution is thereafter used in a packaging system for the storage of ophthalmic devices intended for direct contact with body tissue or body fluid. As used herein, the term “ophthalmic device” refers to devices that reside in or on the eye. These lenses can provide optical correction, wound care, drug delivery, diagnostic functionality or cosmetic enhancement or effect or a combination of these properties. Representative examples of such devices include, but are not limited to, soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking. Any material known to produce an ophthalmic device including a contact lens can be used herein.

The ophthalmic devices can be any material known in the art capable of forming an ophthalmic device as described above. In one embodiment, an ophthalmic device includes devices which are formed from material not hydrophilic per se. Such devices are formed from materials known in the art and include, by way of example, polysiloxanes, perfluoropolyethers, fluorinated poly(meth)acrylates or equivalent fluorinated polymers derived, e.g., from other polymerizable carboxylic acids, polyalkyl(meth)acrylates or equivalent alkylester polymers derived from other polymerizable carboxylic acids, or fluorinated polyolefins, such as fluorinated ethylene propylene polymers, or tetrafluoroethylene, preferably in combination with a dioxol, e.g., perfluoro-2,2-dimethyl-1,3-dioxol. Representative examples of suitable bulk materials include, but are not limited to, Lotrafilcon A, Neofocon, Pasifocon, Telefocon, Silafocon, Fluorsilfocon, Paflufocon, Silafocon, Elastofilcon, Fluorofocon or Teflon AF materials, such as Teflon AF 1600 or Teflon AF 2400 which are copolymers of about 63 to about 73 mol % of perfluoro-2,2-dimethyl-1,3-dioxol and about 37 to about 27 mol % of tetrafluoroethylene, or of about 80 to about 90 mol % of perfluoro-2,2-dimethyl-1,3-dioxol and about 20 to about 10 mol % of tetrafluoroethylene.

In another embodiment, an ophthalmic device includes a device which is formed from material hydrophilic per se, since reactive groups, e.g., carboxy, carbamoyl, sulfate, sulfonate, phosphate, amine, ammonium or hydroxy groups, are inherently present in the material and therefore also at the surface of an ophthalmic device manufactured therefrom. Such devices are formed from materials known in the art and include, by way of example, polyhydroxyethyl acrylate, polyhydroxyethyl methacrylate (HEMA), polyvinyl pyrrolidone (PVP), polyacrylic acid, polymethacrylic acid, polyacrylamide, polydimethylacrylamide (DMA), polyvinyl alcohol and the like and copolymers thereof, e.g., from two or more monomers selected from hydroxyethyl acrylate, hydroxyethyl methacrylate, N-vinyl pyrrolidone, acrylic acid, methacrylic acid, acrylamide, dimethyl acrylamide, vinyl alcohol and the like. Representative examples of suitable bulk materials include, but are not limited to, Polymacon, Tefilcon, Methafilcon, Deltafilcon, Bufilcon, Phemfilcon, Ocufilcon, Focofilcon, Etafilcon, Hefilcon, Vifilcon, Tetrafilcon, Perfilcon, Droxifilcon, Dimefilcon, Isofilcon, Mafilcon, Nelfilcon, Atlafilcon and the like. Examples of other suitable bulk materials include balafilcon A, hilafilcon A, alphafilcon A, hilafilcon B and the like.

In another embodiment, an ophthalmic device includes a device which is formed from materials which are amphiphilic segmented copolymers containing at least one hydrophobic segment and at least one hydrophilic segment which are linked through a bond or a bridge member.

It is particularly useful to employ biocompatible materials herein including both soft and rigid materials commonly used for ophthalmic lenses, including contact lenses. In general, non-hydrogel materials are hydrophobic polymeric materials that do not contain water in their equilibrium state. Typical non-hydrogel materials comprise silicone acrylics, such as those formed from a bulky silicone monomer (e.g., tris(trimethylsiloxy)silylpropyl methacrylate, commonly known as “TRIS” monomer), methacrylate end-capped poly(dimethylsiloxane)prepolymer, or silicones having fluoroalkyl side groups (polysiloxanes are also commonly known as silicone polymers).

Hydrogels in general are a well-known class of materials that comprise hydrated, crosslinked polymeric systems containing water in an equilibrium state. Accordingly, hydrogels are copolymers prepared from hydrophilic monomers. In the case of silicone hydrogels, the hydrogel copolymers are generally prepared by polymerizing a monomeric mixture containing at least one device-forming silicone-containing monomer and at least one device-forming hydrophilic monomer. Either the silicone-containing monomer or the hydrophilic monomer can function as a crosslinking agent (a crosslinker being defined as a monomer having multiple polymerizable functionalities) or a separate crosslinker may be employed. Silicone hydrogels typically have a water content between about 10 to about 80 weight percent.

Representative examples of useful hydrophilic monomers include, but are not limited to, amides such as N,N-dimethylacrylamide and N,N-dimethylmethacrylamide; cyclic lactams such as N-vinyl-2-pyrrolidone; and (meth)acrylated poly(alkene glycols), such as poly(diethylene glycols) of varying chain length containing monomethacrylate or dimethacrylate end caps. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277, the disclosures of which are incorporated by reference herein. Other suitable hydrophilic monomers will be apparent to one skilled in the art. For example, 2-hydroxyethylmethacrylate (HEMA) is a well-known hydrophilic monomer that may be used in admixture with the aforementioned hydrophilic monomers.

The monomeric mixtures may also include a second device-forming monomer including a copolymerizable group and a reactive functional group. The copolyermizable group is preferably an ethylenically unsaturated group, such that this device-forming monomer copolymerizes with the hydrophilic device-forming monomer and any other device-forming monomers in the initial device-forming monomer mixture. Additionally, the second monomer can include a reactive functional group that reacts with a complementary reactive group of the copolymer which is the reaction product of one or more polymerizable polyhydric alcohols and one or more polymerizable fluorine-containing monomers. In other words, after the device is formed by copolymerizing the device-forming monomer mixture, the reactive functional groups provided by the second device-forming monomers remain to react with a complementary reactive moiety of the copolymer.

In one embodiment, reactive groups of the second device-forming monomers include epoxide groups. Accordingly, second device-forming monomers are those that include both an ethylenically unsaturated group (that permits the monomer to copolymerize with the hydrophilic device-forming monomer) and the epoxide group (that does not react with the hydrophilic device-forming monomer but remains to react with the copolymer is the reaction product of one or more polymerizable polyhydric alcohols and one or more polymerizable fluorine-containing monomers). Examples include glycidyl methacrylate, glycidyl acrylate, glycidyl vinylcarbonate, glycidyl vinylcarbamate, 4-vinyl-1-cyclohexene-1,2-epoxide and the like.

As mentioned, one class of ophthalmic device substrate materials are silicone hydrogels. In this case, the initial device-forming monomeric mixture further comprises a silicone-containing monomer. Applicable silicone-containing monomeric materials for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995. Specific examples of suitable materials for use herein include those disclosed in U.S. Pat. Nos. 5,310,779; 5,387,662; 5,449,729; 5,512,205; 5,610,252; 5,616,757; 5,708,094; 5,710,302; 5,714,557 and 5,908,906, the contents of which are incorporated by reference herein.

Representative examples of applicable silicone-containing monomers include bulky polysiloxanylalkyl(meth)acrylic monomers. An example of a bulky polysiloxanylalkyl(meth)acrylic monomer is represented by the structure of Formula IV:

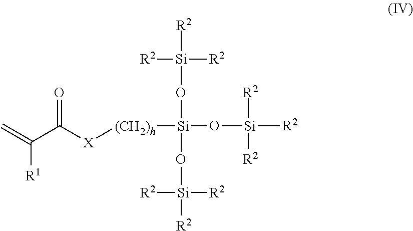

wherein X denotes —O— or —NR— wherein R denotes hydrogen or a C₁ to C₄ alkyl; each R¹ independently denotes hydrogen or methyl; each R² independently denotes a lower alkyl radical such as a C₁ to C₄ alkyl, a phenyl radical or a group represented by

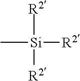

wherein each R^{2′} independently denotes a lower alkyl radical such as a C₁ to C₄ alkyl or a phenyl radical; and h is 1 to 10.

Examples of bulky monomers are methacryloxypropyl tris(trimethyl-siloxy)silane or tris(trimethylsiloxy)silylpropyl methacrylate, sometimes referred to as TRIS and tris(trimethylsiloxy)silylpropyl vinyl carbamate, sometimes referred to as TRIS-VC and the like.

Such bulky monomers may be copolymerized with a silicone macromonomer, which is a poly(organosiloxane) capped with an unsaturated group at two or more ends of the molecule. U.S. Pat. No. 4,153,641 discloses, for example, various unsaturated groups such as acryloxy or methacryloxy groups.

Another class of representative silicone-containing monomers includes, for example, silicone-containing vinyl carbonate or vinyl carbamate monomers such as, for example, 1,3-bis[4-vinyloxycarbonyloxy)but-1-yl]tetramethyl-di siloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; trimethylsilylmethyl vinyl carbonate and the like and mixtures thereof.

Another class of silicone-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference herein. Further examples of silicone urethane monomers are represented by Formulae V and VI:

E(*D*A*D*G)ₐ*D*A*D*E′; or (V)

E(*D*G*D*A)ₐ*D*A*D*E′; or (VI)

wherein:

D independently denotes an alkyl diradical, an alkyl cycloalkyl diradical, a cycloalkyl diradical, an aryl diradical or an alkylaryl diradical having 6 to about 30 carbon atoms;

G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;

* denotes a urethane or ureido linkage;

a is at least 1;

A independently denotes a divalent polymeric radical of Formula VII:

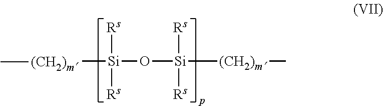

wherein each R^{s} independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;

each of E and E′ independently denotes a polymerizable unsaturated organic radical represented by Formula VIII:

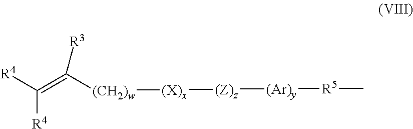

wherein: R³ is hydrogen or methyl;
R⁴ is hydrogen, an alkyl radical having 1 to 6 carbon atoms, or a —CO—Y—R⁶ radical wherein Y is —O—, —S— or —NH—;
R⁵ is a divalent alkylene radical having 1 to about 10 carbon atoms;
R⁶ is a alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

In one embodiment, a silicone-containing urethane monomer is represented by Formula IX:

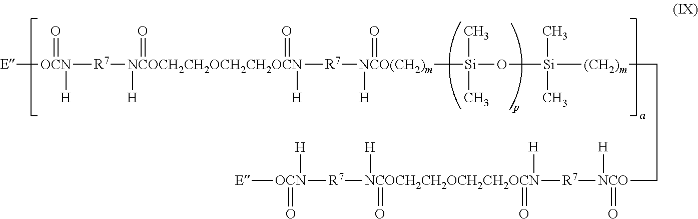

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

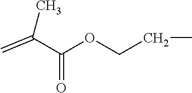

In another embodiment, a silicone hydrogel material comprises (in bulk, that is, in the monomer mixture that is copolymerized) about 5 to about 50 percent, or from about 10 to about 25, by weight of one or more silicone macromonomers, about 5 to about 75 percent, or about 30 to about 60 wt. % of one or more polysiloxanylalkyl (meth)acrylic monomers, and about 10 to about 50 wt. %, or about 20 to about 40 wt. % of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also useful substrates. The silane macromonomer may be a silicone-containing vinyl carbonate or vinyl carbamate or a polyurethane-polysiloxane having one or more hard-soft-hard blocks and end-capped with a hydrophilic monomer.

Another class of representative silicone-containing monomers includes fluorinated monomers. Such monomers have been used in the formation of fluorosilicone hydrogels to reduce the accumulation of deposits on contact lenses made therefrom, as disclosed in, for example, U.S. Pat. Nos. 4,954,587; 5,010,141 and 5,079,319. Also, the use of silicone-containing monomers having certain fluorinated side groups, i.e., —(CF₂)—H, have been found to improve compatibility between the hydrophilic and silicone-containing monomeric units. See, e.g., U.S. Pat. Nos. 5,321,108 and 5,387,662.

The above silicone materials are merely exemplary, and other materials for use as substrates and have been disclosed in various publications and are being continuously developed for use in contact lenses and other medical devices can also be used. For example, an ophthalmic device can be formed from at least a cationic monomer such as cationic silicone-containing monomer or cationic fluorinated silicone-containing monomers.

Ophthalmic devices such as contact lenses for use in the packaging systems of the illustrative embodiments described herein can be manufactured employing various conventional techniques, to yield a shaped article having the desired posterior and anterior lens surfaces. Spincasting methods are disclosed in U.S. Pat. Nos. 3,408,429 and 3,660,545; and static casting methods are disclosed in U.S. Pat. Nos. 4,113,224, 4,197,266 and 5,271,876. Curing of the monomeric mixture may be followed by a machining operation in order to provide a contact lens having a desired final configuration. As an example, U.S. Pat. No. 4,555,732 discloses a process in which an excess of a monomeric mixture is cured by spincasting in a mold to form a shaped article having an anterior lens surface and a relatively large thickness. The posterior surface of the cured spincast article is subsequently lathe cut to provide a contact lens having the desired thickness and posterior lens surface. Further machining operations may follow the lathe cutting of the lens surface, for example, edge-finishing operations.

Typically, an organic diluent is included in the initial monomeric mixture in order to minimize phase separation of polymerized products produced by polymerization of the monomeric mixture and to lower the glass transition temperature of the reacting polymeric mixture, which allows for a more efficient curing process and ultimately results in a more uniformly polymerized product. Sufficient uniformity of the initial monomeric mixture and the polymerized product is of particular importance for silicone hydrogels, primarily due to the inclusion of silicone-containing monomers which may tend to separate from the hydrophilic comonomer.

Suitable organic diluents include, for example, monohydric alcohols such as C₆ to C₁₀ straight-chained aliphatic monohydric alcohols, e.g., n-hexanol and n-nonanol such as 1-nonanol, 2-nonanol and mixtures thereof; diols such as ethylene glycol; polyols such as glycerin; ethers such as diethylene glycol monoethyl ether; ketones such as methyl ethyl ketone; esters such as methyl enanthate; and hydrocarbons such as toluene. In one illustrative embodiment, an organic diluent is sufficiently volatile to facilitate its removal from a cured article by evaporation at or near ambient pressure.

Generally, the organic diluent may be included in an amount of from about 5 to about 60 percent by weight of the monomeric mixture. In one embodiment, the diluent may be included at about 10 to about 50 percent by weight of the monomeric mixture. If necessary, the cured lens may be subjected to solvent removal, which can be accomplished by evaporation at or near ambient pressure or under vacuum. An elevated temperature can be employed to shorten the time necessary to evaporate the diluent.

Following removal of the organic diluent, the lens can then be subjected to mold release and optional machining operations. The machining step includes, for example, buffing or polishing a lens edge and/or surface. Generally, such machining processes may be performed before or after the article is released from a mold part. As an example, the lens may be dry released from the mold by employing vacuum tweezers to lift the lens from the mold.

As one skilled in the art will readily appreciate, ophthalmic device surface functional groups of the ophthalmic device may be inherently present at the surface of the device. However, if the ophthalmic device contains too few or no functional groups, the surface of the device can be modified by known techniques, for example, plasma chemical methods (see, for example, WO 94/06485), or conventional functionalization with groups such as —OH, —NH₂ or —CO₂H. Suitable ophthalmic device surface functional groups of the biomedical device include a wide variety of groups well known to the skilled artisan. Representative examples of such functional groups include, but are not limited to, hydroxy groups, amino groups, carboxy groups, carbonyl groups, aldehyde groups, sulfonic acid groups, sulfonyl chloride groups, isocyanato groups, carboxy anhydride groups, lactone groups, azlactone groups, epoxy groups and groups being replaceable by amino or hydroxy groups, such as halo groups, or mixtures thereof. In one embodiment, the ophthalmic device surface functional groups of the ophthalmic device are amino groups and/or hydroxy groups.

In one embodiment, the foregoing ophthalmic devices are subjected to an oxidative surface treatment such as corona discharge or plasma oxidation followed by treatment with the aqueous packaging solution described herein. For example, an ophthalmic device such as a silicone hydrogel formulation containing hydrophilic polymers, such as poly(N,N-dimethylacrylamide) or poly(N-vinylpyrrolidinone), is subjected to an oxidative surface treatment to form at least silicates on the surface of the lens and then the lens is treated with an aqueous packaging solution as described herein to render a lubricious, stable, highly wettable surface coating. The complexation treatment is advantageously performed under autoclave conditions (sterilization conditions).

Following any surface treatment, the ophthalmic device such as a lens will be immersed in the aqueous packaging solution described above and stored in a packaging system. Generally, a packaging system for the storage of an ophthalmic device includes at least a sealed container containing one or more unused ophthalmic devices immersed in an aqueous packaging solution. In one embodiment, the sealed container is a hermetically sealed blister-pack, in which a concave well containing an ophthalmic device such as a contact lens is covered by a metal or plastic sheet adapted for peeling in order to open the blister-pack. The sealed container may be any suitable generally inert packaging material providing a reasonable degree of protection to the lens, preferably a plastic material such as polyalkylene, PVC, polyamide, and the like.

The method of packaging and storing an ophthalmic device such as a contact lens includes at least packaging an ophthalmic device immersed in the aqueous packaging solution described above. The method may include immersing the ophthalmic device in the aqueous packaging solution prior to delivery to the customer/wearer, directly following manufacture of the contact lens. Alternately, the packaging and storing in the packaging solution may occur at an intermediate point before delivery to the ultimate customer (wearer) but following manufacture and transportation of the lens in a dry state, wherein the dry lens is hydrated by immersing the lens in the packaging solution. Consequently, a package for delivery to a customer may include a sealed container containing one or more unused contact lenses immersed in an aqueous packaging solution and heat sterilized according to the present invention.

In one embodiment, the steps leading to the present ophthalmic device packaging system includes (1) molding an ophthalmic device in a mold comprising at least a first and second mold portion, (2) hydrating and cleaning the device in a container which may comprise at least one of the mold portions, (3) introducing the packaging solution with the copolymer into the container with the device supported therein, and (4) sealing the container. The method further includes the step of heat sterilizing the contents of the container. Sterilization may take place prior to, or most conveniently after, sealing of the container and may be affected by any suitable method known in the art. In one illustrative embodiment. heat sterilizing is carried out by autoclaving the sealed container at temperatures of about 120° C. or higher. During the heat sterilization process, the polyquaternium polymer in the aqueous packaging solution decomposes into its monomeric and oligomeric species thereby providing a preservative-free, sterile packaged ophthalmic device product. This generally occurs by a hydrolysis reaction at a carbon bonded to the quaternary ammonium nitrogen atom by either an S_{N}2 or S_{N}1 mechanism.

The following examples are provided to enable one skilled in the art to practice the invention and are merely illustrative. The examples should not be read as limiting the scope of the illustrative embodiments as defined in the claims. In the examples, the following abbreviations are used.

The polyquaternium polymer used in the examples was Polyquaternium-1 (hereinafter “PQ-1”) represented by the following structure:

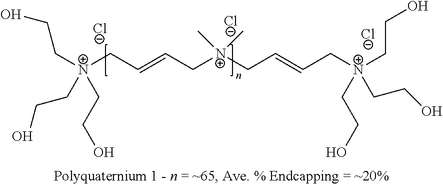

The molecular weight of the PQ-1 was 6680 g/mol not including the chloride counterions, and 9055 g/mole including the chloride counterions.]

### Example 1

An aqueous packaging solution was made by mixing the following components, listed in Table 1 at amounts per weight.

TABLE 1
Solution A

| Ingredient | % w/w |
|---|---|
| Sodium Phosphate | 0.00925 |
| Monobasic Monohydrate | |
| Sodium Phosphate | 0.0320 |
| Dibasic Anhydrous | |
| Potassium Chloride | 0.70 |
| Poloxamine 1107 | 0.550 |
| Poloxamer 181 | 0.020 |
| Glycerol | 0.90 |
| Erythritol | 0.90 |
| Polyquaternium-1 | Various |
| Purified Water | Q.S.to 100% w/w |

An initial preservative efficacy (PE) screen was carried out on different concentrations of PQ-1 ranging from 0 ppm to 10 ppm in Solution A, both non-autoclaved and autoclaved. The autoclave sterilization was carried out at 121° C. for 30 minutes. The PE screen utilized only the bacterial organisms specified in ISO 14730. The purpose of the screen was to show that PQ-1 preservative efficacy is lost after one autoclave cycle and also to narrow the concentration range of PQ-1 for full organism PE testing according to EN ISO 14730:2014. The organisms tested in the screen included Staphylococcus aureus (S.a.), Pseudomonas aeruginosa (P.a.) and Escherichia coli (E.c.). The results of the PE screen are shown below in Tables 2 and 3.

TABLE 2
Day 14 PE Screen of Various Concentrations
of PQ-1 in Solution A

| Concentration of PQ-1 in Solution A | S.a. | P.a. | E.c. |
|---|---|---|---|
| Solution A with 0 ppm, Autoclaved | 0.7 | −0.5 | 0.1 |
| Solution A with 1 ppm, Autoclaved | 1 | −0.2 | −0.1 |
| Solution A with 5 ppm, Autoclaved | >4.7 | −0.5 | 0.2 |
| Solution A with 10 ppm, Autoclaved | >4.7 | −0.3 | 0.2 |
| Solution A with 0 ppm, Not Autoclaved | >4.7 | −0.5 | 0.4 |
| Solution A with 1 ppm, Not Autoclaved | >4.7 | >4.6 | >4.7 |
| Solution A with 5 ppm, Not Autoclaved | >4.7 | >4.6 | >4.7 |
| Solution A with 10 ppm, Not Autoclaved | >4.7 | >4.6 | >4.7 |

TABLE 3
Day 28 PE Screen of Various Concentrations
of PQ-1 in Solution A

| Concentration of PQ-1 in Solution A | S.a. | P.a. | E.c. |
|---|---|---|---|
| Solution A with 0 ppm, Autoclaved | 0.6 | −0.4 | 0.5 |
| Solution A with 1 ppm, Autoclaved | 0.9 | −0.3 | 0.4 |
| Solution A with 5 ppm, Autoclaved | >4.7 | −0.5 | 1 |
| Solution A with 10 ppm, Autoclaved | >4.7 | −0.4 | 0.9 |
| Solution A with 0 ppm, Not Autoclaved | >4.7 | −0.4 | 1.1 |
| Solution A with 1 ppm, Not Autoclaved | >4.7 | >4.6 | >4.7 |
| Solution A with 5 ppm, Not Autoclaved | >4.7 | >4.6 | >4.7 |
| Solution A with 10 ppm, Not Autoclaved | >4.7 | >4.6 | >4.7 |

None of the solutions which had been autoclaved passed the PE screen criteria of a minimum of 3 log reduction of each bacteria in 14 days. There was no re-challenge at day 14 in this screen so the 28 day results only reflect the result of an additional 2 weeks of static soaking.

### Example 2

Since all three of the non-autoclaved PQ-1 solutions passed the preservative efficacy screen criteria in Example 1 for the bacteria and all were unpreserved after autoclaving, both the 1 ppm and 5 ppm concentrations of PQ-1 were selected as the candidates for full PE testing against the EN ISO 14730 criteria which additionally includes a yeast (Candida albicans, C.a.) and a mold (Aspergillus brasiliensis, A.b.) as test organisms.

PE testing was carried out on both the non-autoclaved and autoclaved samples using Solution A of Table 1 containing either 1 ppm or 5 ppm PQ-1. The autoclave sterilization was carried out at 121° C. for 30 minutes. Following standard EN ISO 14730:2014, a rechallenge step was conducted after 14 days using an inoculum level of 1.0×10⁴ cfu/mL to 1.0×10⁵ cfu/mL for each organism. The results of PE testing are shown below in Tables 4 and 5.

TABLE 4
Day 14 PE Results of 1 ppm and 5 ppm PQ-1 in Solution A

| Concentration of PQ-1 in Solution A | S.a. | P.a. | E.c. | C.a. | A.b. |
|---|---|---|---|---|---|
| Solution A with 1 ppm, | >4.8 | −0.4 | 0.6 | 0.0 | −0.3 |
| Autoclaved | | | | | |
| Solution A with 5 ppm, | >4.8 | −0.3 | 0.1 | −0.1 | −0.3 |
| Autoclaved | | | | | |
| Solution A with 1 ppm, | >4.8 | >4.8 | >4.8 | 2.2 | −0.3 |
| Not Autoclaved | | | | | |
| Solution A with 5 ppm, | >4.8 | >4.8 | >4.8 | 2.9 | −0.3 |
| Not Autoclaved | | | | | |

TABLE 5
Day 28 PE Results after Rechallange of PQ-1 in Solution A

| Concentration of PQ-1 in Solution A | S.a. | P.a. | E.c. | C.a. | A.b. |
|---|---|---|---|---|---|
| Solution A with 1 ppm, | >3.8 | 0.1 | 0.6 | 0.2 | −0.1 |
| Autoclaved | | | | | |
| Solution A with 5 ppm, | >3.8 | −0.2 | 0.5 | 0.0 | −0.1 |
| Autoclaved | | | | | |
| Solution A with 1 ppm, | >3.8 | >3.9 | >3.8 | 1.3 | −0.1 |
| Not Autoclaved | | | | | |
| Solution A with 5 ppm, | >3.8 | >3.9 | >3.8 | 0.7 | 0.0 |
| Not Autoclaved | | | | | |

Both of the 1 ppm and 5 ppm PQ-1 in Solution A met the full requirements of EN ISO 14730:2014 before autoclaving. After autoclaving the solutions were unpreserved.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A method, comprising:
(a) adding at least one polyquaternium polymer to an aqueous packaging solution for preventing antimicrobial contamination during storage;
(b) packaging the aqueous packaging solution and an ophthalmic device in a manner preventing contamination of the ophthalmic device by one or more microorganisms; and
(c) heat sterilizing the aqueous packaging solution and the ophthalmic device, whereby the at least one polyquaternium polymer decomposes during the heat sterilizing to obtain a preservative-free packaged ophthalmic device product.

2. The method of claim 1, wherein the ophthalmic device is a contact lens.

3. The method of claim 1, wherein the polyquaternium polymer comprises from about 30 to about 50,000 quaternary-amine-functional repeating units.

4. The method of claim 1, wherein the polyquaternium polymer has a weight average molecular weight Mw of about 3,000 to about 5,000,000.

5. The method of claim 1, wherein the polyquaternium polymer has a weight average molecular weight Mw of about 5,000 to about 40,000.

6. The method of claim 3, wherein the polyquaternium polymer is cationic.

7. The method of claim 1, wherein the polyquaternium polymer is present in the aqueous packaging solution in an amount of about 0.1 ppm to about 100 ppm.

8. The method of claim 1, wherein the aqueous packaging solution further comprises:
about 0.01 wt. % to about 5.0 wt. %, based on the total weight of the aqueous packaging solution, of erythritol;
about 0.01 wt. % to about 1.0 wt. %, based on the total weight of the aqueous packaging solution, of the one or more poloxamer comfort agents; and
about 0.1 wt. % to about 3.0 wt. %, based on the total weight of the aqueous packaging solution, of glycerol.

9. The method of claim 1, wherein the aqueous packaging solution further comprises a poloxamine.

10. The method of claim 1, wherein the aqueous packaging solution further comprises a poloxamine, sodium chloride, potassium chloride, a phosphate buffer or any combination thereof.

11. The method of claim 1, wherein the aqueous packaging solution further comprises a buffer agent, a tonicity adjusting agent, a cleaning agent, a wetting agent, a nutrient agent, a sequestering agent, a viscosity builder, a contact lens conditioning agent, an antioxidant or any combination thereof.

12. The method of claim 1, wherein heat sterilizing comprises autoclaving the aqueous packaging solution and the ophthalmic device at a temperature of at least about 120° C.

13. The method of claim 1, wherein the aqueous packaging solution does not contain an effective disinfecting amount of a disinfecting agent.

14. The method of claim 1, wherein the aqueous packaging solution does not contain a germicide compound.

15. The method of claim 1, wherein the aqueous packaging solution contains a borate buffer in an amount below 0.3 wt. %.

16. The method of claim 1, wherein the aqueous packaging solution does not contain a borate buffer.

17. A method, comprising:
(a) immersing an ophthalmic device in an aqueous packaging solution comprising at least one polyquaternium polymer antimicrobial agent, wherein the aqueous packaging solution has an osmolality of at least about 150 mOsm/kg and a pH in the range of about 6 to about 9;
(b) packaging the aqueous packaging solution and the ophthalmic device in a manner minimizing contamination of the ophthalmic device by one or more microorganisms; and
(c) heat sterilizing the aqueous packaging solution and the ophthalmic device to obtain a preservative-free, sterile package ophthalmic device product.

18. The method of claim 17, wherein the ophthalmic device is a contact lens.

19. The method of claim 17, wherein the polyquaternium polymer comprises from about 30 to about 50,000 quaternary-amine-functional repeating units.

20. The method of claim 17, wherein the polyquaternium polymer has a weight average molecular weight Mw of about 3,000 to about 5,000,000.

21. The method of claim 17, wherein the polyquaternium polymer has a weight average molecular weight Mw of about 5,000 to about 40,000.

22. The method of claim 19, wherein the polyquaternium polymer is cationic.

23. The method of claim 17, wherein the polyquaternium polymer is present in the aqueous packaging solution in an amount of about 0.1 ppm to about 100 ppm.

24. The method of claim 17, wherein the aqueous packaging solution further comprises:
about 0.01 wt. % to about 5.0 wt. %, based on the total weight of the aqueous packaging solution, of erythritol;
about 0.01 wt. % to about 1.0 wt. %, based on the total weight of the aqueous packaging solution, of the one or more poloxamer comfort agents; and
about 0.1 wt. % to about 3.0 wt. %, based on the total weight of the aqueous packaging solution, of glycerol.

25. The method of claim 17, wherein the aqueous packaging solution further comprises a poloxamine, sodium chloride, potassium chloride, one or more phosphate buffers or any combination thereof.

26. The method of claim 17, wherein heat sterilizing comprises autoclaving the aqueous packaging solution and the ophthalmic device at a temperature of at least about 120° C.

27. The method of claim 17, wherein the aqueous packaging solution contains a borate buffer in an amount below 0.3 wt. %.
